(19) United States
(12) Patent Application Publication
(10) Pub. No.: US 2008/0006264 A1
(43) Pub. Date: Jan. 10, 2008

(54) Method and device for cleaning the nebuliser membrane in an inhalation therapy device

(75) Inventors: Thomas Gallem, Munchen (DE); Uwe Hetzer, Munchen (DE); Philipp Holzmann, Munchen (DE)

Correspondence Address: WOLF GREENFIELD & SACKS, P.C., 600 ATLANTIC AVENUE, BOSTON, MA, 02210-2206, US

(73) Assignee: Pari GmbH Spezialisten fur effektive Inhalation, Starnberg (DE)

(21) Appl. No.: 11/824,827

(22) Filed: Jul. 3, 2007

(30) Foreign Application Priority Data
Jul. 4, 2006 (DE) DE 102006030833.6

(51) Int. Cl. A61M 11/00

(52) U.S. Cl. 128/200.14; 422/20; 422/28

(57) ABSTRACT

The invention describes a device and a method for cleaning an oscillatable membrane (3) of a membrane aerosol generator (4) of an inhalation therapy device, in which a cleaning liquid is supplied to the membrane (3) on the aerosol side, and the membrane (3) is caused to oscillate in such a manner that the cleaning liquid is conveyed through the openings of the membrane (3) to the liquid side of said membrane.

The invention relates to a method and a device for cleaning the membrane of a membrane aerosol generator of an inhalation therapy device.

Using inhalation therapy devices, therapeutically effective or medicament-containing liquids are nebulised into an aerosol consisting of respirable particles by means of an aerosol generator. The generated aerosol is offered to a patient for inhalation during an inhalation therapy, as a result of which the therapeutically effective liquid or medicament reaches the airways of the patient.

In particularly effective inhalation therapy devices, a membrane aerosol generator is used as the aerosol generator for generating the aerosol. The liquid to be nebulised is stored in a storage container and is supplied to the membrane of the membrane aerosol generator on the one side, i.e. the liquid side. If the membrane of the membrane aerosol generator is caused to oscillate, the liquid is transported through the openings of the membrane and is released on the other side of the membrane, i.e. the aerosol side, in the form of an aerosol. To cause the membrane to oscillate, the membrane aerosol generator comprises, in addition to the membrane, an oscillation generator, for example a ring-shaped piezo-electric element and a ring-shaped substrate that are connected to one another and to the membrane in such a manner that a control signal supplied to the piezo-electric element triggers oscillation of the oscillation generator, which causes the membrane to oscillate.

As an example of such an inhalation therapy device, EP 1 304 131 describes an inhalation therapy device having an oscillatable membrane for nebulising a liquid, comprising an oscillation generating device having at least one connecting means for supplying a control signal, by means of which the membrane is caused to oscillate such that a liquid disposed on one side of the membrane is nebulised through the membrane and is present on the other side of the membrane as an aerosol.

Inhalation therapy devices with membrane aerosol generators are characterised by a high dosage accuracy and short therapy times. High-quality aerosols can be generated in particular owing to the excellent controllability of aerosol generation, and thus inhalation therapy devices having membrane aerosol generators are particularly suitable for the application of very expensive and/or highly effective medicaments. In addition, inhalation therapy devices with membrane aerosol generators can be used over a long period of time with satisfactory results. After a large number of therapy sessions, aerosol generation may become slower, however this does not lead to a deterioration of the aerosol quality.

In almost all cases of use, an excessive extension of the therapy sessions is not acceptable since a rapid administration of a specific dose of the medicament is always desired. This is because a patient/user of the inhalation therapy device should not have to put up with unnecessary therapy times. Accordingly, long therapy times are basically undesirable. Furthermore, the comparatively high manufacturing costs of high-quality inhalation therapy devices with membrane aerosol generators are to be taken into consideration. There is thus the need to further extend the lifespan of the inhalation therapy device, i.e. the period of time over which an inhalation therapy device with a membrane aerosol generator can be used without a clear deterioration of the properties relevant for therapy.

In view of the above, the technical problem to be solved by the invention is to extend the period of time over which an inhalation therapy device with a membrane aerosol generator can be used with a high quality level.

This problem is solved by means of a method according to claim 1 and a device according to claim 4. Advantageous embodiments are specified in each of the sub-claims.

The solution according to the invention resulted from examinations using a scanning electron microscope of membranes used in membrane aerosol generators. These examinations showed that in spite of a regular and thorough cleaning of the membrane aerosol generator, an increasingly large number of holes occasionally becomes blocked by material of different origins, as a result of which the speed of aerosol generation is reduced, which has a negative effect on the duration of the therapy session.

The cleaning of the membrane of the membrane aerosol generator carried out to date does not prevent or eliminate the now observed obstruction of individual openings of the membrane to the same extent as the cleaning according to the invention, even if selected cleaning agents such as solvents are used in the known methods. The known types of cleaning are effective, though not to the extent now achieved by the invention.

The blockages are only removed by supplying the cleaning liquid to the aerosol side of the membrane and activating membrane oscillations that transport the cleaning liquid through the membrane so that the cleaning liquid drips off on the liquid side of the membrane. In other words, an effective cleaning of the membrane is achieved by a hitherto unknown backwashing mode of the membrane in which a cleaning liquid flows through the membrane in a direction opposite to the direction of aerosol generation. The membrane of the membrane aerosol generator is thereby caused to oscillate and thus assumes an active role in the cleaning process as compared to previously known cleaning processes in which the cleaning liquid or the manner of supply of the cleaning liquid assumes an active role.

The present invention will be explained in more detail in the following with reference to the figures, in which:

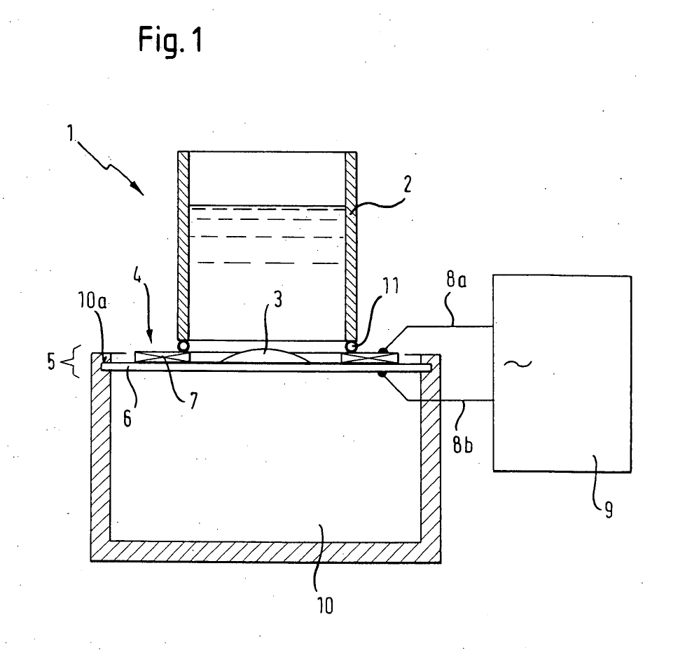

FIG. 1 shows a first embodiment of a device for cleaning an oscillatable membrane of an inhalation therapy device according to the invention;

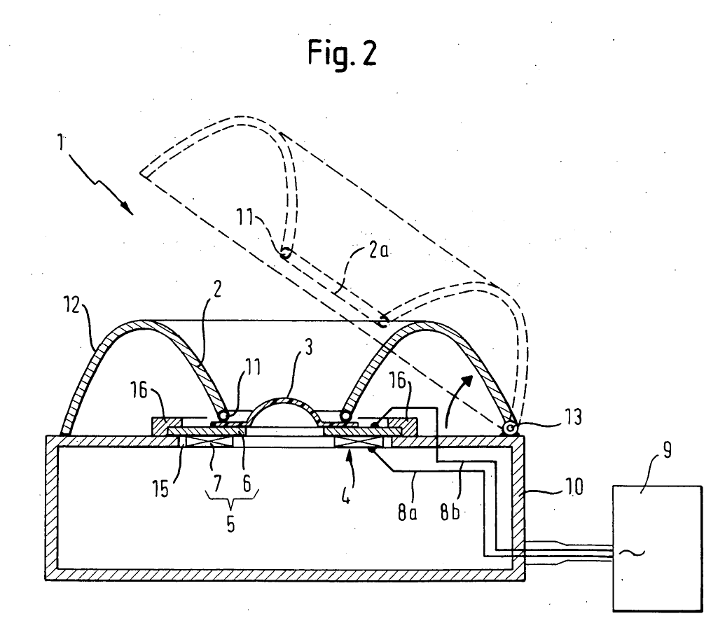

FIG. 2 shows a second embodiment of the cleaning device according to the invention of an inhalation therapy device according to the invention;

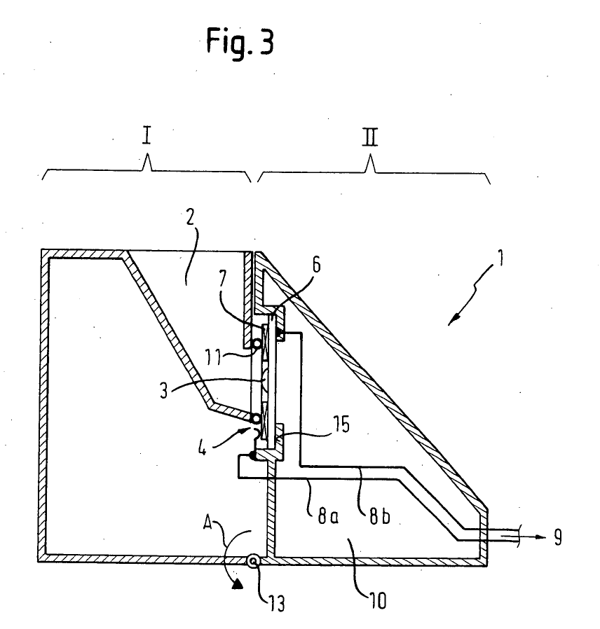

FIG. 3 shows a third embodiment of a cleaning device according to the invention of an inhalation therapy device according to the invention;

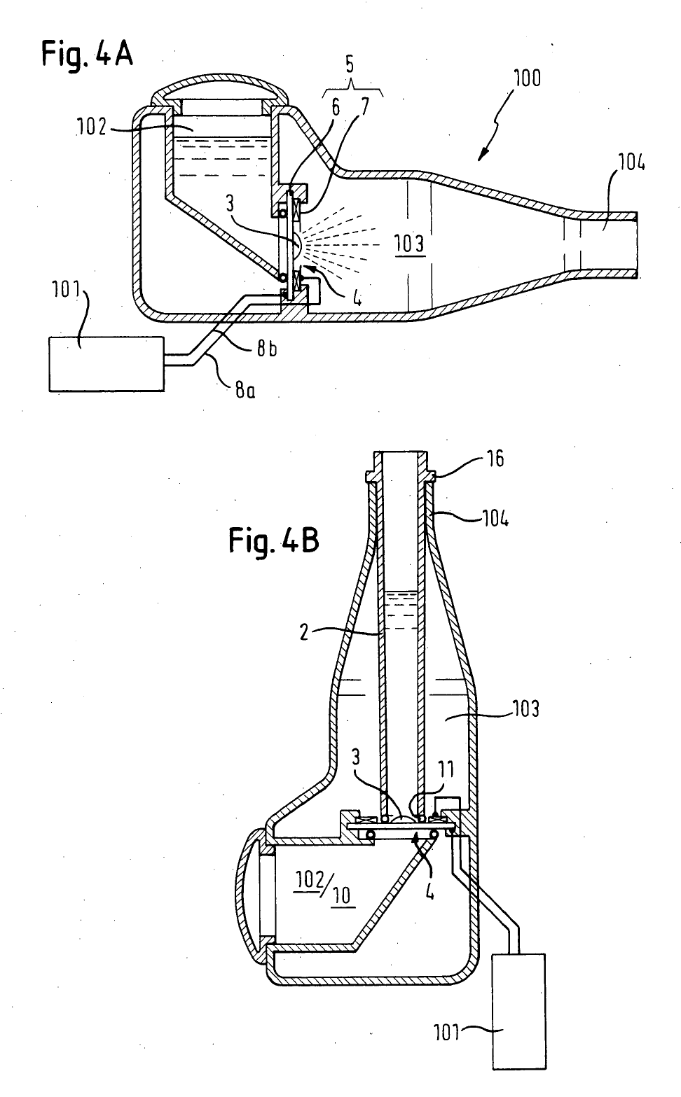

FIG. 4A shows an inhalation therapy device with a membrane aerosol generator;

FIG. 4B shows a fourth embodiment of the cleaning device according to the invention of an inhalation therapy device according to the invention.

FIG. 1 shows a first advantageous embodiment of a device according to the invention for cleaning an oscillatable membrane of a membrane aerosol generator of an inhalation therapy device. The shown cleaning device 1 comprises a liquid supply means 2 for supplying a cleaning liquid to the aerosol side of a membrane 3. According to the invention, the cleaning liquid is supplied to that side of the membrane 3 on which the aerosol generated by the membrane aerosol generator is usually released during nebulisation of a liquid.

In addition to the oscillatable membrane 3, the membrane aerosol generator 4 comprises an oscillation generator 5 that consists, in the shown embodiment, of a ring-shaped substrate 6 and a ring-shaped piezo-electric element 7 connected therewith. In the shown embodiment, the oscillation generator 5 thus has a rotationally symmetrical basic structure whose axis of rotation lies in the drawing plane of FIG. 1.

As is furthermore shown by FIG. 1, connecting lines 8a, 8b are on the one hand connected with the oscillation generator 5 and, in accordance with the invention, are on the other hand connected with an oscillation activating means 9 for activating oscillations of the membrane.

According to the embodiment shown in FIG. 1, the oscillation activating means 9 supplies an electric control signal to the oscillation generator 5 of the membrane aerosol generator 4. When the control signal is supplied, the oscillatable membrane 3 is caused to oscillate by the oscillation generator 5 and the cleaning liquid is conveyed, according to the invention, from the aerosol side of the membrane 3 through the membrane 3 to the liquid side of the membrane 3.

As is shown by FIG. 1, the cleaning liquid conveyed through the openings of the membrane is advantageously collected in a collecting receptacle 10, on which the membrane aerosol generator 4 is suitably arranged or attached. For example, according to FIG. 1, the ring-shaped substrate 6 is fixed at its outer periphery in a groove 10a of the collecting receptacle 10.

In the embodiment shown in FIG. 1, the liquid supply means 2 is realised in the form of a hollow cylinder that is open at both front ends. One of the front ends of the hollow cylinder 2 is positioned such that this front end is tightly sealed by the membrane 3 or the membrane aerosol generator 4. A ring-shaped seal 11 is furthermore advantageously arranged on the front end facing the membrane, said seal abutting the membrane 3 or the membrane aerosol generator 4 in such a manner that a cleaning liquid filled into the hollow cylinder does not escape at the front end of the hollow cylinder facing the membrane but is rather disposed on the membrane 3. Provided that the hollow cylinder has a sufficient weight of its own to obtain the leak tightness (with or without seal 11) to be achieved for supplying the cleaning liquid, further measures for fixing the liquid supply means 2 are not necessary. However, fixings for the liquid supply means 2 can be alternatively (or additionally) provided, which reliably hold the liquid supply means 2 in the position suitable for supplying the cleaning liquid.

FIG. 2 shows a second advantageous embodiment of a cleaning device according to the invention. In the cleaning device 1 shown in FIG. 2, the liquid supply means 2 for supplying a cleaning liquid to the aerosol side of the membrane 3 is formed in the shape of a funnel. Furthermore, the funnel-shaped liquid supply means 2 is configured integrally with a lid 12 that is fixed to the collecting receptacle 10 so as to be pivotable about an axis 13. In the closed state, the funnel-shaped liquid supply means 2 is positioned such that the free open end 2a abuts the membrane 3 or the membrane aerosol generator 4 so that a cleaning liquid filled into the funnel-shaped liquid supply means 2 is disposed on the membrane 3. A seal 11 is advantageously provided at the free open end of the liquid supply means 2 in order to prevent an undesirable escape of the cleaning liquid from the volume limited by the liquid supply means 2 and the membrane 3.

So that the membrane aerosol generator 4 can be removed from and inserted in the cleaning device 1 according to the second embodiment, the liquid supply means 2 can be pivoted together with the lid 12 about the axis 13. The membrane aerosol generator 4 is detachably fixed to an accommodating member 15 provided in the collecting receptacle, for example an adapted opening in the surface of the collecting receptacle 10 facing the lid, by means of one or more holding devices 16 that, for example, clamp the membrane aerosol generator 4.

As in the first embodiment, supply of the cleaning liquid in the second embodiment also occurs, in accordance with the invention, to the aerosol side of the membrane 3, i.e. to that side on which the aerosol generated by the membrane aerosol generator is usually released during nebulisation of a liquid. If the membrane aerosol generator 4 is activated and the membrane 3 is thus caused to oscillate, the cleaning liquid exits on the other side of the membrane 3, i.e. on that side on which the liquid to be nebulised is normally disposed in the nebulising mode.

As in the first embodiment, a control signal is supplied to the membrane aerosol generator 4 by an oscillation activating means 9 via connecting lines 8a and 8b.

FIG. 3 shows a third embodiment of a cleaning device 1 according to the invention. In this embodiment, the membrane aerosol generator 4 is arranged vertically rather than horizontally in the cleaning device in order to demonstrate that the method according to the invention can also be carried out if the membrane 3 of the membrane aerosol generator is not arranged horizontally. The membrane 3 of the membrane aerosol generator 4 can ultimately be arranged as desired provided that the cleaning liquid is supplied to the aerosol side of the membrane 3 of the membrane aerosol generator 4 with the help of the liquid supply means 2 and the membrane 3 is caused to oscillate such that the cleaning liquid is conveyed from the aerosol side to the liquid side of the membrane 3.

In the third embodiment shown in FIG. 3, the liquid supply means 2 is a container that is slanted in parts and has a vertical opening which is arranged such that the cleaning liquid is disposed on the aerosol side of the membrane 3. A seal 11 is preferably provided at the opening of the liquid supply means 2, which prevents an undesirable escape of the cleaning liquid at the site of contact between funnel/seal and membrane/membrane aerosol generator.

As is indicated by the arrow A in FIG. 3, part I of the cleaning device 1, in which the liquid supply means 2 is formed, can be pivoted about a rotational axis, which exposes the accommodating member 15 for the membrane aerosol generator 4 that is formed in another part II of the cleaning device 1. The membrane aerosol generator 4 is placed in the accommodating member 15 and is thereby connected with connecting lines 8a and 8b, via which a control signal is introduced to the membrane aerosol generator 4. When the first housing part I of the cleaning device 1 is pivoted into the position shown in FIG. 3, the membrane aerosol generator 4 or the membrane 3 is fixed in the accommodating member since the seal 11 acts mechanically on the membrane aerosol generator 4. The second housing part II of the cleaning device 1 according to FIG. 3 serves as a collecting chamber 10 for the cleaning liquid conveyed through the membrane.

FIG. 4A shows an inhalation therapy device 100 with a membrane aerosol generator 4, which comprises, in addition to the membrane 3, an oscillation generator having a ring-shaped substrate 6 and a ring-shaped piezo-electric element 7 that are connected with one another and with the membrane 3 in such a manner that the membrane 3 is caused to oscillate if the oscillation generator 5 is stimulated to oscillate by an electric control signal. The electric control signal is supplied via supply lines 8a, 8b that are connected to a control device 101 of the inhalation therapy device 100. The liquid to be nebulised is filled into a liquid reservoir 102 of the inhalation therapy device and is disposed on the liquid side of the membrane 3 in the nebulising mode. If a control signal is supplied to the membrane aerosol generator 4 by the control device 101, the membrane 3 is caused to oscillate, and the liquid to be nebulised is thus transported from the liquid side to the aerosol side of the membrane 3. The aerosol is released by the membrane aerosol generator 4 into a mixing chamber 103, from which a patient inhales the aerosol via a mouthpiece 104. Even though the mouthpiece 104 is shown integrally in FIG. 4A, it can, in many cases, be removed from the mixing chamber 103.

In an inhalation therapy device configured in this manner, the cleaning device according to the invention comprises, as is shown in FIG. 4B, a liquid supply means 2 that is in the form of a tube that is inserted into the mouthpiece 104 of the inhalation therapy device. The length of the liquid supply means 2 depends on the size of the mouthpiece 104 and the mixing chamber 103 and is configured to be long enough that an open front end of the liquid supply means 2 reaches the membrane 3 such that the seal 11 provided on this front end abuts the membrane 3 or the membrane aerosol generator 4. If the mouthpiece 104 can be removed from the mixing chamber 103, the dimensions of the tube-like liquid supply means 2 can also be adapted to the mixing chamber 103.

The liquid supply means 2 preferably has a stop 16 that abuts the opening edge of the mouthpiece 104 or the mixing chamber 103 of the inhalation therapy device and thus prevents the liquid supply means 2 from being inserted too far into the inhalation therapy device 100, thereby damaging the membrane 3 or the membrane aerosol generator 4. Furthermore, the tube 2 is preferably tapered so as to facilitate insertion into the mouthpiece or the mixing chamber and to obtain at one end a front end cross-section that is adapted to the membrane 3 or the membrane aerosol generator 4 and to obtain at the opposite end a front end cross-section that is adapted to the opening of the mouthpiece 104 or the mixing chamber 103.

In the tube 2 that forms the liquid supply means of the cleaning device according to the invention in the fourth embodiment, the cleaning liquid is filled in such that it is disposed on the aerosol side of the membrane 3 during the cleaning process. The membrane 3 is caused to oscillate by the control device 101, which is also used to control the membrane aerosol generator 4 for the generation of an aerosol. The control device 101 of the inhalation therapy device 100 is thus advantageously also used for cleaning. If a control signal is supplied to the membrane aerosol generator 4 by the control device 101, the membrane 3 is caused to oscillate, and thus the cleaning liquid supplied to the aerosol side of the membrane 3 with the help of the liquid supply means 2 is transported through the membrane 3 and collected in the liquid reservoir 102. The liquid reservoir 102 of the inhalation therapy device 100 thereby serves as a collecting receptacle 10 for the cleaning liquid conveyed through the membrane 3. However, in order to prevent the cleaning liquid collected in the liquid reservoir 102 being inadvertently nebulised and inhaled, the lid of the liquid reservoir 102 should preferably be removed during cleaning. In order to collect the cleaning liquid then exiting the liquid reservoir, a separate collecting receptacle 10 must be provided. In this manner, the further advantage that no cleaning liquid is inadvertently nebulised, which is inherent to all the embodiments according to the invention, is also reliably achieved in the embodiment shown in FIG. 4B.

It is of particular advantage in the last described fourth embodiment of a cleaning device according to the invention that the cleaning liquid is supplied to the aerosol side of the membrane since it is prevented in this manner that a cleaning liquid filled into the liquid reservoir is inadvertently nebulised and inhaled. Supplying the cleaning liquid to the aerosol side of the membrane prevents the inhalation therapy device possibly being used to nebulise a liquid.

Furthermore, the fourth embodiment is advantageous because the control device 101 of the inhalation therapy device 100 is used as an oscillation activating means 9 according to the invention in order to cause the membrane 3 to oscillate so that the cleaning liquid is transported from the aerosol side to the liquid side. The control device of an inhalation therapy device can also be used in the embodiments described beforehand to supply the control signal to the membrane aerosol generator so that the membrane is caused to oscillate in order to effect the desired transport of the cleaning liquid from the aerosol side to the liquid side of the membrane. However, in a cleaning device according to the invention, a control means that is independent of the inhalation therapy device can also be used as an oscillation activating means, which provides the opportunity to supply other control signals, for example in other frequency ranges or with other waveforms. However, it has been shown that good cleaning results are achieved if the control device of the inhalation therapy device is used under the operating conditions that are also used for nebulisation of the liquid in order to generate an aerosol.

A highly effective method for cleaning an oscillatable membrane 3 of a membrane aerosol generator 4 of an inhalation therapy device can be carried out with a cleaning device according to the invention. Furthermore, a cleaning liquid is supplied according to the invention to the aerosol side of the membrane 3. This means that supply occurs on that side of the membrane on which the generated aerosol exits in the normal nebulising mode of the membrane aerosol generator. Furthermore, the membrane 3 is caused to oscillate according to the invention in such a manner that the cleaning liquid is conveyed through the openings of the membrane 3 to the liquid side of the membrane.

Distilled water, ethanol or a solvent or another suitable liquid can be used as the cleaning liquid.

According to the invention, activation of the membrane oscillations preferably occurs at the frequency that is also used for nebulising a liquid in the normal nebulising mode. However, control can also take place in another manner such that the control signal supplied during cleaning of the membrane differs from the control signal of a nebulising mode, in particular as regards amplitude, frequency and frequency change during cleaning, duration and sequence of the activation periods.

1. Method for cleaning an oscillatable membrane of a membrane aerosol generator of an inhalation therapy device, wherein:
a cleaning liquid is supplied to the membrane on an aerosol side, and
the membrane is caused to oscillate such that the cleaning liquid is conveyed through openings of the membrane to a liquid side of said membrane.

2. Method according to claim 1, wherein the cleaning liquid is distilled water, ethanol, a solvent or a mixture hereof or other cleaning liquids.

3. Method according to claim 1, wherein the oscillations are caused in the same manner as the membrane is caused to oscillate in the nebulising mode.

4. Device for cleaning an oscillatable membrane of a membrane aerosol generator of an inhalation therapy device comprising:
a liquid supply means for supplying a cleaning liquid to an aerosol side of the membrane, and
an oscillation activating means for activating membrane oscillations such that the cleaning liquid is conveyed through openings of the membrane to a liquid side of said membranes.

5. Device according to claim 4, wherein the liquid supply means is a hollow cylinder that is arranged with one front end on the membrane or the membrane aerosol generator such that a cleaning liquid filled into the hollow cylinder is disposed on said membranes.

6. Device according to claim 5, wherein the hollow cylinder is configured as an elongated tube that is designed for insertion into a mouthpiece or a mixing chamber of an inhalation therapy device.

7. Device according to claim 6, wherein the elongated tube has a stop that limits insertion of the tube into the mouthpiece of an inhalation therapy device.

8. Device according to claim 6, wherein the elongated tube extends in a tapered manner towards the end provided for arrangement on the membrane.

9. Device according to claim 4, wherein the liquid supply means is funnel-shaped and is arranged with an opening on the membrane or the membrane aerosol generator in such a manner that a cleaning liquid filled into the funnel-shaped liquid supply means is disposed on the membrane.

10. Device according to claim 4, wherein a seal is provided on the liquid supply means, which prevents the escape of the cleaning liquid at the membrane or the membrane aerosol generators.

11. Device according to claim 4, wherein a collecting receptacle is provided for collecting the cleaning liquid exiting on the liquid side of the membrane.

12. Device according to claim 10, wherein an accommodating member for the arrangement of the membrane aerosol generator is provided in the collecting receptacle.

13. Device according to claim 11, wherein the liquid supply means and the collecting receptacle are configured so as to be pivotable about an axis in relation to one another.

14. Device according to claim 13, wherein the liquid supply means is configured integrally with a lid.

15. Device according to claim 4, wherein the oscillation activating means is a control means that emits a preferably electric control signal to control the membrane aerosol generator.

16. Device according to claim 15, wherein the control means is the control device of the inhalation therapy device.

17. Device according to claim 15, wherein the control signal of the oscillation activating means corresponds to the control signal that is supplied to the membrane aerosol generator in a nebulising mode.

18. Device according to claim 15, wherein the control signal of the oscillation activating means differs from the control signal of a nebulising mode in particular as regards amplitude, frequency and frequency change during cleaning, duration and sequence of the activation periods.
